(19) United States
(12) Patent Application Publication
(10) Pub. No.: US 2006/0127390 A1
(43) Pub. Date: Jun. 15, 2006

(54) Enzymatic inactivation of transmissible spongiform encephalopathy agents and dectection there of

(76) Inventors: Donald L. Hoglund, North Raleigh, NC (US); Terry J. Arver, Menomonie, WI (US)

Correspondence Address: NOVOZYMES NORTH AMERICA, INC., 500 FIFTH AVENUE, SUITE 1600, NEW YORK, NY, 10110, US

(21) Appl. No.: 11/191,847

(22) Filed: Jul. 29, 2005

Related U.S. Application Data
(60) Provisional application No. 60/593,005, filed on Jul. 30, 2004.
(60) Provisional application No. 60/600,497, filed on Aug. 10, 2004.
(60) Provisional application No. 60/600,498, filed on Aug. 10, 2004.

(51) Int. Cl. A61K 38/48

(52) U.S. Cl. 424/94.64

(57) ABSTRACT

The invention relates to a method for inactivating transmissible spongiform encephalopathy agents by contact with a subtilisin protease. The invention further relates to detection of any residual transmissible spongiform encephalopathy agents.

### CROSS-REFERENCE TO RELATED APPLICATIONS

This application claims priority or the benefit under 35 U.S.C. 119 of U.S. provisional application Nos. 60/593,005, 60/600,497 and 60/600,498 filed Jul. 30, 2004, Aug. 10, 2004 and Aug. 10, 2004, the contents of which are fully incorporated herein by reference.

### TECHNICAL FIELD

The present invention relates to a method for inactivating transmissible spongiform encephalopathy agents with an enzyme and optionally detection thereof.

### BACKGROUND OF THE INVENTION

Prion diseases are some of the most intriguing infectious disorders affecting the brains of humans and animals. The prevalent hypothesis proposes that the infectious agent is a misfolded protein that propagates in the absence of nucleic acid by transmission of its altered folding to the normal host version of the protein. Prion diseases, also called transmissible spongiform encephalopathies (TSEs), are a group of infectious neurodegenerative disorders affecting humans and animals. These diseases are characterized by brain vacuolation, astrogliosis, neuronal apoptosis and accumulation of the misfolded, protease-resistant prion protein (PrPres) in the central nervous system. The transmissibility of TSEs was accidentally demonstrated in 1937, when a population of Scottish sheep was inoculated against a common virus with a formalin extract of brain tissue unknowingly derived from an animal with scrapie. Stunningly, after 2 years, nearly 10% of the flock developed scrapie. Scrapie was subsequently transmitted experimentally to sheep and mice. In humans, an infectious route was suspected for the propagation of the TSE kuru among the cannibalistic tribes of New Guinea, and this was demonstrated in 1966. This was followed by observations of transmission to animals of Creutzfeld Jacobs disease (CJD) and a familial form of TSE, Gerstmann-Straussler-Scheinker syndrome (GSS).

There is a need for methods for inactivation of transmissible spongiform encephalopathy agent

There is a need for methods for inactivation of transmissible spongiform encephalopathy agent in human and animal tissue which may have been contaminated with such agents, e.g. slaughterhouse waste material. Recent experiments cast doubt on the efficacy of chemical treatments such as sodium hypochlorite or sodium hydroxide which were previously thought to completely inactivate infectivity. US 2004/0091474 (Raven) discloses a method for inactivation of a transmissible spongiform encephalopathy agent comprising exposing the transmissible spongiform encephalopathy agent to a thermostable proteolytic enzyme. This method is carried out at temperatures equal to or greater than 40° C.

### SUMMARY OF THE INVENTION

The inventors have found that transmissible spongiform encephalopathy agents can be inactivated by contact with a subtilisin protease and that this method is effective at temperatures below 40° C. Accordingly the present invention relates to a method for inactivating a transmissible spongiform encephalopathy agent comprising contacting the transmissible spongiform encephalopathy agent with a subtilisin protease.

### DETAILED DISCLOSURE OF THE INVENTION

The invention generally relates to methods for the destruction of prions; e.g. plant, such as yeast, prions and mammalian prions whether infectious and/or naturally occurring hereinafter referred to variously as prions, aggregated prions, infectious prions, plant, such as yeast, prions or tissue, and mammal prions or tissue. More specifically, the invention relates to the application of enzymes at low temperatures to mammalian tissues, in animal products derived from mammalian tissues, on surfaces, and in liquids, and other places, hereinafter referred to variously as “material” where infectious prions of mammalian origin and/or prions of plant origin may reside providing for at least a reduction by destruction in mammal prion and yeast prion content of said material. The invention destroys mammalian prion protein and yeast prion protein as determined by Bio-Rad Enzyme Linked Immunosorbant Assay (ELISA) and the inability of ELISA or Bio-Rad Western blot immunodiagnostics to reveal said prion protein after said protein is subjected to the methods and materials and processes of the invention.

The present invention in some embodiments is based on, but not limited to, the use of multiple enzymes and enzyme types, multiple other substances, in varying combinations and concentrations, at varying times and temperatures, and under various environmental conditions, providing the degradation of prions respective of certain pH levels, and in some embodiments at pH levels less than about 10; yeast prion protein and mammal prion protein as it relates to exposing the prion protein from wherever it resides, comprising locations such as, but not limited to, surfaces, in liquids, on tissue, in tissue, within cells of tissue, in extra-cellular space, and in organelles such as exosomes in the mammalian system. The invention is in some embodiments preferably conducted in conditions of pH, and in some embodiments less than about 10, with one enzyme or multiple enzymes, or in multiple steps, incorporating one or more enzymes and other substances, and under varying environmental conditions. The invention provides methods and composition for destruction of prions; plant, such as yeast, prion protein and mammal prion protein, infectious or otherwise, wherever plant prion protein and mammal prion protein may reside or exist outside the live animal or in the environment. The invention relates to inactivation of a transmissible spongiform encephalopathy agent.

In one embodiment the invention relates to a method for inactivating a transmissible spongiform encephalopathy agent comprising contacting the transmissible spongiform encephalopathy agent with a protease of the subtilisin family. By the term transmissible spongiform encephalopathy agent is meant an agent capable of infecting a human or other mammal with transmissible spongiform encephalopathy, such as e.g. with human Creutzfeld Jacobs disease, variant human Creutzfeld Jacobs disease, Kuru, bovine spongiform encephalopathy, chronic wasting disease, and sheep scrapie. In one embodiment the transmissible spongiform encephalopathy agent is an infectious prion, i.e. a prion which, in contrast to a normal non-infectious prion, is capable of infecting a human or other mammal with transmissible spongiform encephalopathy.

Contacting a transmissible spongiform encephalopathy agent with subtilsin protease may be conducted at any suitable temperature. In one embodiment of the invention a transmissible spongiform encephalopathy agent is contacted with subtilsin protease at a temperature below 30° C., such as below 25° C. or below 20° C. In another embodiment of the invention a transmissible spongiform encephalopathy agent is contacted with subtilsin protease at a temperature between 0° C. and 29° C., such as between 0° C. and 25° C., between 0° C. and 20° C., between 2° C. and 25° C., or between 4° C. and 20° C. In a further embodiment of the invention a transmissible spongiform encephalopathy agent is contacted with subtilsin protease at a temperature between 0° C. and 89° C., such as between 4° C. and 55° C. pH during contact of a transmissible spongiform encephalopathy agent with subtilsin protease according to the invention may be any suitable pH at which the selected subtilisin protease is active. In one embodiment a transmissible spongiform encephalopathy agent is contacted with subtilsin protease at a pH between 2 and 10, such as between 4 and 8, between 6 and 8, between 6.1 and 7.9, or between 6.5 and 7.9. The duration of contact of a transmissible spongiform encephalopathy agent with subtilsin protease according to the invention may be any duration suitable to obtain desired inactivation of the transmissible spongiform encephalopathy agent. In one embodiment the duration of contact of a transmissible spongiform encephalopathy agent with subtilsin protease according to the invention is between 5 minutes and 80 hours, such as between 1 hour and 72 hours, between 4 hours and 48 hours, or between 4 hours and 24 hours.

The method of the invention may be useful in cleaning and sanitation of objects or materials that are suspected of being contaminated with a transmissible spongiform encephalopathy agent, or are potentially contaminated with a transmissible spongiform encephalopathy agent. Such objects or materials may e.g. be carcasses, bones, knives, bone saws, saw blades, splitting saw blades, housing for splitting saw, hoses, tables, containers, wash rooms, floors, carcass washing cabinets, chains, tackles for chains, ropes, floors, walls, floor drains, screens, clothing, hard hats, truck floors, surfaces, containers, e.g. containers of plastic or metal, vats, rubber boots, gloves, and/or protective eyewear. Accordingly, in one embodiment the invention relates to a method for inactivating a transmissible spongiform encephalopathy agent wherein an object or material potentially contaminated with transmissible spongiform encephalopathy agent is contacted with subtilisin protease. An object or material potentially contaminated with transmissible spongiform encephalopathy agent may be contacted with subtilisin protease by any suitable method, e.g. by contacting the object or material with a liquid solution of subtilisin protease, e.g. by submersion of the object or material in the liquid solution or by spraying the liquid solution onto he object or material A liquid solution of subtilisin protease may further comprise additional components useful for cleaning of objects or materials, such as e.g. surfactants, which may be non-ionic including semi-polar and/or anionic and/or cationic and/or zwitterionic. Examples of surfactants are e.g. linear alkylbenzenesulfonate, alpha-olefinsulfonate, alkyl sulfate (fatty alcohol sulfate), alcohol ethoxysulfate, secondary alkanesulfonate, alpha-sulfo fatty acid methyl ester, alkyl- or alkenylsuccinic acid or soap. In one embodiment of the invention an object or material potentially contaminated with transmissible spongiform encephalopathy agent is submerged in a liquid solution of subtilisin protease. In another embodiment a liquid solution of subtilisin protease is sprayed onto an object or material potentially contaminated with transmissible spongiform encephalopathy agent. In still another embodiment an object or material potentially contaminated with transmissible spongiform encephalopathy agent is contacted with a mixture of subtilisin protease and one or more surfactants.

Subtilisin Protease

Subtilisin proteases are extracellular serine endopeptidases belonging to peptidase family S8 and classified as EC 3.4.21.62 in the enzyme nomenclature system set out by the Nomenclature Committee of the International Union of Biochemistry and Molecular Biology (IUBMB). Subtilisins are characterised by a common catalytic triad of aspartate-histidine-serine reading from the amino to carboxy terminal of the amino acid sequence. A subtilisin protease according to the invention may be obtained from any source, e.g. microbial source, such as bacterial or fungal source. The term “obtained from” as used herein in connection with a source of subtilisin protease, means that the subtilisin is produced by the specific source, or by a cell in which a gene from the source has been inserted. In one embodiment of the invention a subtilisin protease is a subtilisin variant. In the context of this invention, the term subtilisin variant means a subtilisin that has been produced by an organism which is expressing a mutant gene derived from a parent organism which possessed an original or parent gene and which produced a corresponding parent enzyme, the parent gene having been mutated in order to produce the mutant gene from which said subtilisin variant is produced when expressed in a suitable host.

In one embodiment of the invention a subtilisin protease is a subtilisin obtained from a Bacillus species, e.g. B. subtilis, B. amyloliquefaciens, B. licheniformis, B. lentus, B. alcalophilus, B. natto, B. clausii, or B. stearothermophilus, or a variant thereof. In a further embodiment a subtilisin protease is Subtilisin 309 from Bacillus clausii (old name: Bacillus lentus C360). In another embodiment a subtilisin protease is Subtilisin A from Bacillus licheniformis. In still another embodiment a subtilisin protease is a variant of Subtilisin 309 from Bacillus clausii wherein valine at position 66 of Subtilisin 309 from Bacillus clausii has been substituted with alanine, and serine at position 104 has been substituted with alanine, using the amino acid sequence and numbering of Subtilisin 309 from Bacillus clausii (former classified as B. lentus) given in Sequence No. 4 of WO 03/006602, which is hereby incorporated by reference.

A transmissible spongiform encephalopathy agent to be treated according to the invention may be present in mammalian tissue. In one embodiment of the invention mammalian tissue, cells, sub-cellular materials, or yeast tissue is subjected to grinding to a particle size sufficient to liquefy the tissue into a slurry. In a further embodiment mammalian tissue is grinded to a size of below 10 millimetres before contact with a subtilisin protease, such as below 6 millimetres, e.g. 4-6 millimetres. In a still further embodiment tissue, surfaces, or liquids potentially contaminated with infectious prions are heated to sufficient, uniform temperature by, but not limited to; natural heat, by water baths, by steam to temperatures from about 20° C. to about 89° C., and in some embodiments from about 0° C. to about 20° C., for a time of about contact to 24 hours.

In some embodiments of the invention tissue, surfaces, or liquids may be heated sufficiently to between approximately 20° C. to about 89° C., and in some embodiments to between approximately 0° C. to about 20° C., for a sufficient time of about 10 seconds to 1 hour, in order that the enzymes in this invention can work within a suitable range of temperature on tissue that is uniformly heated. The uniformly heated tissue in containers is cooled by processes, but not limited to, air cooled at room temperature, refrigerated, or placed in a cool water bath and the temperature is lowered to between 20° C. and 89° C., and in some embodiments to between 0° C. and about 20° C., and then the tissue, surface, or liquid is exposed to proprietary enzymes concentrations at proprietary times, combinations, and concentrations for a time of between contact to about 24 hours.

Specified risk materials may be processed by the method of the invention, which might, comprise, but not be limited to: the brain, skull, eyes, trigeminal ganglia, spinal cord, vertebral column (excluding the vertebrae of the tail, the transverse processes of the thoracic and lumbar vertebrae, and the wings of the sacrum), and dorsal root ganglia of cervids, ovine, murine species and other bovine 30 months and older; and the tonsils and distal ileum of the small intestine of all cattle and any cattle showing central nervous system disease or are unable to ambulate on their own. Specified risk materials should be considered constantly broadening as the disease of TSE is broadening, as would be appreciated by one of ordinary skill in the art.

A further aspect of the invention relates to methods and processes of degrading prion protein material, both naturally occurring and infectious, consisting of (a) heating material containing prion material or prion material alone to a temperature in a first elevated temperature range from about 20° C. to about 89° C., and in some embodiments range from about 0° C. to about 20° C., and then (b) cooling said material to a lower elevated temperature range from about 20° C. to 89° C., and in some embodiments a lower temperature range from about 0° C. to about 20° C., and (c) then exposing said material to the methods and processes of the invention including exposing the material to the proprietary enzymes and enzyme concentrations contained in the invention that are effective at such lower temperature ranges from about contact to 24 hours and thus rendering the material into a material containing a reduced amount of prion protein, both mammal and yeast, both infectious and naturally occurring.

A still further application of the invention relates to a method of at least partially degrading the prion content of a material containing prion protein, both naturally occurring and infectious prion protein, by the steps and methods and processes of the invention, including heating the material to a range from about 20° C. to about 89° C., and in some embodiments to a range from about 0° C. to about 20° C., and simultaneously exposing the material to the methods and processes of the invention and to the proprietary enzymes and combinations of enzymes, at a time from about contact to 24 hours to a least partially degrade the prion protein, both naturally occurring and infectious prion protein.

In another particular aspect, the invention relates to methods and processes of processing animal tissues and animal byproducts, hereinafter referred to as material, to destroy at least some of the prion material, both naturally occurring and infectious, and thus removing therefrom, by exposing the material to the proprietary enzymes contained in this invention for a sufficient time under sufficient temperature to destroy at least some of the prion protein in the material.

A process or combination of process that may or may not include heating mammal or plant tissue (materials)—whether in small microscopic amounts or in large tonnages, on potential contaminated surfaces or in liquids, and in some embodiments specified risk materials, or in the environment at large prior to exposure to the processes, materials, enzymes, chemicals and devices of this invention, (1) may or may not include washing, homogenizing, adding substances or molecules to or removing substances or molecules from, (2) calibrating amount of said materials, (3) exposing the materials to the processes or combination of processes to various times and temperatures, and evaluating the materials after exposure with a combination of one or more quality control devices. If heating occurs of a material or a surface or a liquid, washing may or may not occur, drying may or may not occur, enzymes singly or in combination may be placed with the material for a time from the point of contact to 24 hours.

In some embodiment the invention relates to a method of treatment of tissue, surfaces, and liquids for destruction of prion protein in the following steps:

(a) providing said tissue, surface, or liquid in a form in which prion protein is accessible for enzymatic exposure;

(b) heating the tissue to a temperature in a range of about 20° C. to about 89° C., and in some embodiments to a temperature in a range of about 0° C. to about 20° C., for a sufficient time to enhance enzymatic susceptibility of said prion protein therein;

(c) exposing the heated tissue, surface, or liquid to Bacillus Licheniformis Carlsberg protease at a temperature in a range of about 20° C. to about 89° C., and in some embodiments at a temperature in a range of about 0° C. to about 20° C., for a sufficient time; and (d) verifying destruction of infective prion protein in the tissue, on surfaces, or in the liquid.

In further embodiments the invention relates to a method of treatment of tissue, surfaces, and liquids for destruction of prion protein in the following steps:

(a) providing said tissue, surface, or liquid in a form in which prion protein is accessible for enzymatic exposure;

(b) exposing the tissue, surface, or liquid to Bacillus Licheniformis Carlsberg protease at a temperature in a range of about 20° C. to about 89° C., and in some embodiments at a temperature in a range of about 0° C. to about 20° C., for a sufficient time to degrade the prion protein; and

(c) verifying destruction of infective prion protein in the tissue, on surfaces, or in the liquid.

In still further embodiments the invention relates to a method to treatment of tissue, surface, or liquid for reduction of prion protein, including infectious prion protein therein, the method comprising the steps of:

(a) providing said tissue, surface, or liquid in a form in which infectious prion protein is accessible for enzymatic exposure;

(b) heating the tissue to a temperature in a range of about 20° C. to about 89° C., and in some embodiments to a temperature in a range of about 0 C. to about 20° C., for a sufficient time to enhance enzymatic susceptibility of said prion protein therein;

(c) exposing the heated tissue, surface, or liquid to Bacillus Licheniformis Carlsberg protease to a temperature in a range of about 20° C. to about 89° C., and in some embodiments to a temperature in a range of about 0 C to about 20° C., for a sufficient time to enhance the proteolytic susceptibility of infectious prion protein; and verifying at least partial reduction of infective prion protein in the tissue, on surfaces, or in the liquid.

In still further embodiments the invention relates to a method of treatment of tissue, surface or liquid for reduction of infective prion protein therein, the method comprising the steps of:

(a) providing said tissue, surface, or liquid in a form in which said infective prion protein is accessible for enzymatic exposure;

(b) heating the tissue, surface, or liquid to a temperature in a range of from about 20° C. to about 89° C., and in some embodiments to a temperature in a range of about 0° C. to about 20° C., for a time sufficient to enhance the susceptibility of infectious prion protein therein; and

(c) exposing the heated tissue to a proteolytic enzyme, or enzymes that are effective for at least partial reduction of the infective prion protein in such tissue, on surfaces, or in liquid.

In still further embodiments the invention relates to a method for reducing infective prion protein in bovine, ovine, cervid, murine, and any other mammal tissue, or surfaces contaminated or suspected of contamination by infectious prions of any kind, by such potentially infected tissue, surfaces or liquids, or liquids contaminated by or suspected of contamination by any substance, surface, or liquid suspected of containing infectious prion protein, herein after referred to as the ‘material’, the method including (a) providing said ‘material’ in a form in which said infectious prion protein therein is accessible for enzymatic exposure, (b) heating the ‘material’ at a temperature in a range about 20° C. to about 89° C., and in some embodiments at a temperature in a range about 0° C. to about 20° C., followed by (c) exposing the ‘material’ to a proteolytic enzyme or enzymes at a temperature in a range of from about 20° C. to about 89° C., and in some embodiments at a temperature in a range of from about 0° C. to about 20° C., at which the enzymes are thermally stable and effective to destroy the infective prion protein associated with prion disease in bovine, ovine, cervid, and murine species, including hamsters and raccoons, wherein said enzymes are selected from a group consisting of subtilisins, lipases, and amylases.

The method of previously described, wherein said heating is conducted for a time of from about contact to 24 hours.

The method previously described in the second preceding paragraph, wherein the proteolytic enzyme comprises Bacillus Licheniformis Carlsberg subtilisin and/or Alcalase.

A method of degrading infectious prion protein contained in animal tissue, on surfaces, or in liquids, comprising (a) homogenizing said animal tissue, or cleaning gross tissues from surfaces and applying the methods and processes of this invention to the gross tissues and surfaces, or degrading prion proteins in liquid filled containers suspected of containing said infectious prion protein, (b) heating the animal tissue, surface, or liquid containing said infectious prion protein to a temperature in a first elevated range of from about 20° C. to about 89° C., and in some embodiments to a temperature in a first elevated range of from about 0° C. to about 20° C., followed by (c) cooling the animal tissue, surface, or liquid containing infectious prion protein to a second temperature that is lower than the first temperature and is in a second elevated temperature range of from about 20° C. to about 89° C., and in some embodiments in a second elevated temperature range of from about 0° C. to about 20° C., and (d) exposing the animal tissue, surface, or liquid containing the infectious prion protein to proteolytic, lypolytic, and glycolytic enzyme(s) effective at such second, lower temperatures to degrade the infectious prion protein, wherein the proteolytic, lypolytic, and glycolytic enzyme(s) inherent to certain processes of this invention are selected from a group of Subtilisins, Lipases, and Amylases in a variable, but prescribe order, under various times from about contact to 24 hours and within various temperatures ranging from about 20° C. to about 89° C., and in some embodiments within various temperatures ranging from about 0° C. to about 20° C., and directed to contain various enzyme concentrations entering homogenous solutions of tissue, on surfaces, or in liquids at various intervals depending on material decontamination need.

An embodiment of this invention, which may comprise one or more features previously described, may alone or in combination with such one or more features comprise processes and other embodiments that protect, limit, reduce, or validate reduction or elimination, and in some embodiments prohibit, reduce or limit cross contamination of prions. The present invention, accordingly, may utilize the features of this invention where needs are clearly defined in, such declarations as the FDA or other government entities may define or describe, and as may be described or defined herein.

The method of the invention may comprise testing for residual transmissible spongiform encephalopathy agent in contact with an object or material potentially contaminated with transmissible spongiform encephalopathy agent after contact with subtilisin protease. Testing for residual transmissible spongiform encephalopathy agent may be performed by any suitable method known in the art, e.g. by ELISA (enzyme-linked immunosorbent assay) as described in the examples following hereafter. Testing may also be performed by subjecting animals susceptible to infection with the transmissible spongiform encephalopathy agent in question to material to be tested for residual transmissible spongiform encephalopathy agent, e.g. by intra-abdominal injection of material, and observing whether the animals develop a disease and/or clinical symptoms caused by the transmissible spongiform encephalopathy agent, and/or by examining tissue, e.g. brain and/or spleen, of the animals for presence of the transmissible spongiform encephalopathy agent and/or signs of pathological changes related to infection with the transmissible spongiform encephalopathy agent. Additionally, testing may be performed by the Misfolded Protein Diagnostic (MPD) assay described in A. Grosset, K. Moskowitz, C. Nelsen, T. Pana, E. Davidson, C. S. Orser. Rapid presymptomatic detection of PrP^{Sc} via conformationally responsive palindromic PrP peptides. Peptides, 2005, prepublished on the internet on 14 Apr. 2005; and/or the test described in Olga Tcherkasskaya, Eugene A. Davidson, Mary Jo Schmerr & Cindy S. Orser. Conformational biosensor for diagnosis of prion diseases. Biotechnology Letters (2005) 27: 671-675.

The processes and other embodiments herein may utilize the distinct features of antibody/antigen reactions, fluorescing antibodies, chemicals whose purpose is to reveal protein existence, and are often used in determining the presence or absence of cross contamination especially on dedicated equipment, such as, but not limited to, deboning equipment, saws, saw blades, knives, cutting surfaces, floors, or tables, at, but not limited to, zoos, feed mills, packing plants, butchers, renderers, or any animal and plant products facilities, or ports-of-entry. This invention embodies enzymatic reduction or destruction of animal and plant prions and a process to validate said reduction or destruction of said prions and processes revealing features such as, but not limited to, immunoflourescence, antigen/antibody reactions, fluorescing antibodies, bio-luminescence, and immunogenic processes and properties. An added feature of this invention is to reduce, limit, and potentially stop all contamination and cross contamination of noninfected environments, materials, tissues, surfaces, or liquids where ever prion contamination may occur by infected prion protein material.

### EXAMPLES

Anson Units (AU)

In the present examples activity of subtilisin proteases is expressed in Anson Units (AU). One Anson Unit (AU) of subtilisin will hydrolyze casein to produce color equivalent to 1.0 micromole (181 micrograms) of tyrosine per min at pH 7.5 at 37° C. (color by Folin-Ciocalteu reagent).

Detection of TSE Agent Inactivation

Presence of TSE agents were detected by ELISA using the BioRad TeSeE™ ELISA (enzyme-linked immunosorbent assay) test kit (Bio-Rad Laboratories, Hercules, Calif., USA) according to the manufacturer's instructions. The test is an immunoenzymatic sandwich test for detection of abnormal prion protein. Samples, as well as positive and negative control samples, were added to wells of a microplate which had been coated with a monoclonal antibody. After incubation the wells were washed, and a secondary monoclonal antibody conjugated with a peroxidase was added. After incubation and washing, a solution containing a peroxidase substrate and a chromogenic indicator was added, and after incubation at specified conditions, the peroxidase reaction was stopped. The color development was determined by measuring optical density (OD) at 450 nm and 620 nm. The cut-off value was determined as the average OD of four negative control samples plus 0.210. Samples with an OD below 90% were considered negative, and samples with an OD between 90% and 100% of the cut-off value were considered suspect and should be retested.

### Example 1

A process in accordance with the present invention may e.g. be performed in the following way: 0.25 mL containing 250 microliters of homogenated deer brain is heated to 80° C. for 5 minutes, cooled to 50° C. for 3 minutes, B. Licheniformis Subtilisin A Carlsberg is added as 250 microliters of a 6% solution contain same to a final volume of 0.5 mL of a 3% solution in a tube and incubated for 5 minutes at 50° C. Heat sample at 80° C. for 5 minutes to stop reaction and Bio-Rad ELISA elected for deer is performed to determine the presence or absence of Chronic Wasting prion material. Optical densities are scanned and read with provided ELISA equipment.

### Example 2

### Comparison of TSE Agent Inactivation with Two Different Subtilisins

Enzymes

Enzyme A: Subtilisin 309 from Bacillus clausii

Enzyme B: A variant of Subtilisin 309 from Bacillus clausii wherein valine at position 66 was substituted with alanine, and serine at position 104 was substituted with alanine. The enzymes were dissolved in phosphate buffers at pH 8 and pH 9 to combine with brain homogenate naturally at pH 7 to yield final pH of 7.5 and 8.0. as indicated, in amounts needed to obtain the indicated activities in the final samples when enzyme solution were mixed with equal amounts of brain homogenate.

Brain Homogenate

Homogenate of cervidae brain from animals infected with Chronic Wasting Disease (CWD) was obtained from Colorado State University Veterinary Diagnostic Lab.

Enzmmatic Treatment

CWD brain homogenate and enzyme solution was mixed in equal amounts and mixed thoroughly with a Vortex mixer. The mixtures were incubated for the indicated time and temperature. After incubation the mixtures were heated to 85 C for 35 minutes to inactivate the enzymes. Presence of CWD infectious agents was detected by BioRad TeSeE™ ELISA as described above. The cut-off value for OD was determined as 0.216. Samples with OD values below 90% of the cut-off value are considered to be negative.

Results

TABLE 1
Measured optical densities (OD) for
samples treated with Enzyme A.

| Incubation conditions | | | Enzyme concentration (%). | | | | | |
|---|---|---|---|---|---|---|---|---|
| | Temp | Time | 1% corresponds to 0.27 AU/ml sample | | | | | |
| pH | (° C.) | (hours) | 0.01 | 0.025 | 0.05 | 0.10 | 0.50 | 1.00 |
| 8.0 | 55 | 4 | 0.475 | 0.230 | 0.088 | 0.017 | 0.014 | 0.012 |
| | | | 0.500 | 0.264 | 0.072 | 0.039 | 0.015 | 0.013 |
| 7.5 | 55 | 4 | 0.525 | 0.286 | 0.122 | 0.026 | 0.014 | 0.009 |
| | | | 0.473 | 0.253 | 0.132 | 0.040 | 0.013 | 0.011 |
| 8.0 | 50 | 14 | 0.156 | 0.033 | 0.022 | 0.023 | 0.016 | 0.015 |
| | | | 0.309 | | 0.020 | 0.021 | 0.016 | |
| 8.0 | 20 | 14 | 0.841 | 0.232 | 0.103 | 0.121 | 0.026 | 0.019 |
| | | | 0.573 | | 0.199 | 0.100 | 0.025 | 0.021 |
| 7.5 | 20 | 14 | 0.544 | 0.593 | 0.357 | 0.129 | 0.057 | 0.032 |
| | | | 0.973 | 0.451 | 0.275 | 0.155 | 0.041 | 0.029 |

TABLE 2
Measured optical densities (OD) for
samples treated with Enzyme B.

| Incubation conditions | | | Enzyme concentration (%). | | | | | |
|---|---|---|---|---|---|---|---|---|
| | Temp | Time | 1% corresponds to 0.27 AU/ml sample | | | | | |
| pH | (° C.) | (hours) | 0.005 | 0.0125 | 0.025 | 0.050 | 0.250 | 0.500 |
| 8.0 | 20 | 14 | 0.653 | 1.124 | 0.835 | 0.455 | 0.091 | 0.044 |
| | | | 1.363 | 1.077 | 0.749 | 0.457 | 0.063 | 0.045 |
| 7.5 | 20 | 14 | 1.192 | 1.159 | 0.745 | 0.611 | 0.180 | 0.058 |
| | | | 1.478 | 1.088 | 0.788 | 0.702 | 0.172 | 0.075 |

### Example 3

### Comparison of TSE Agent Inactivation at 4° C. and 20° C.

Enzyme

Subtilisin A from Bacillus licheniformis (Sigma Chemical Company, St. Louis, USA). The enzyme was dissolved in phosphate buffer at pH 7.5 in amounts needed to obtain the indicated activities in the final samples when enzyme solution were mixed with equal amounts of brain homogenate.

Brain Homogenate

Homogenate of cervidae brain from animals infected with Chronic Wasting Disease (CWD) was obtained from Colorado State University Veterinary Diagnostic Lab.

Enzymatic Treatment

CWD brain homogenate and enzyme solution was mixed in equal amounts and mixed thoroughly with a Vortex mixer. The mixtures were incubated for the indicated time and temperature. After incubation the mixtures were heated to 85 C for 35 minutes to inactivate the enzymes. Presence of CWD infectious agents was detected by BioRad TeSeE™ ELISA as described above. The cut-off value for OD was determined as 0.223. Samples with OD values below 90% of the cut-off value are considered to be negative.

Results

TABLE 3
Measured optical densities (OD) for samples treated
at 4° C. and 20° C., respectively.

| Incubation conditions | | Enzyme concentration (%). | | | |
|---|---|---|---|---|---|
| Temperature | Time | 1% corresponds to 0.27 AU/ml sample | | | |
| (° C.) | (hours) | 0.10 | 0.25 | 0.50 | 1.00 |
| 4 | 24 | 0.030 | 0.014 | 0.023 | 0.008 |
| 4 | 48 | 0.040 | 0.022 | 0.024 | 0.020 |
| 20 | 24 | 0.023 | 0.022 | 0.020 | 0.017 |
| 20 | 48 | 0.038 | 0.021 | 0.023 | 0.015 |

1. A method for inactivating a transmissible spongiform encephalopathy agent comprising contacting the transmissible spongiform encephalopathy agent with a subtilisin protease.

2. The method of claim 1 wherein the transmissible spongiform encephalopathy agent is contacted with the subtilisin protease at a temperature below 30° C.

3. The method of claim 2 wherein the transmissible spongiform encephalopathy agent is contacted with the subtilisin protease at a temperature between 0° C. and 20° C.

4. The method of claim 1 wherein the transmissible spongiform encephalopathy agent is contacted with the subtilisin protease for a time between 5 minutes and 80 hours.

5. The method of claim 1 wherein the transmissible spongiform encephalopathy agent is contacted with the subtilisin protease at a pH between 6 and 8.

6. The method of claim 5 wherein the transmissible spongiform encephalopathy agent is contacted with the subtilisin protease at a pH between 6.5 and 7.9.

7. The method of claim 1 wherein the subtilisin protease is derived from a strain of Bacillus clausii.

8. The method of claim 1 wherein the transmissible spongiform encephalopathy agent is the causative agent of a transmissible spongiform encephalopathy selected from the group consisting of human Creutzfeld Jacobs disease, variant human Creutzfeld Jacobs disease, Kuru, bovine spongiform encephalopathy, chronic wasting disease, and Sheep scrapie.

9. The method of claim 1 comprising contacting mammalian tissue containing a transmissible spongiform encephalopathy agent with a subtilisin protease.

10. A method for reducing infective prion protein in bovine, ovine, cervid, and murine tissue, comprising:
a) providing said tissue in a form in which said infective prion protein therein is accessible for enzymatic exposure;
b) heating said tissue at a temperature in the range from about 0° C. to about 89° C.; and
c) exposing said tissue to a subtilisin protease at a temperature of between about 0° C. and about 20° C. at which the subtilisin protease is thermally stable and effective to destroy the infective prion protein.

11. The method of claim 1 wherein an object or material potentially contaminated with transmissible spongiform encephalopathy agent is contacted with subtilisin protease.

12. The method of claim 11 wherein an object or material potentially contaminated with transmissible spongiform encephalopathy agent is submerged in a liquid solution of subtilisin protease.

13. The method of claim 1 wherein a liquid solution of subtilisin protease is sprayed onto an object or material potentially contaminated with transmissible spongiform encephalopathy agent.

14. The method of claim 11 wherein an object or material potentially contaminated with transmissible spongiform encephalopathy agent is contacted with a mixture of subtilisin protease and one or more surfactants.

15. The method of claim 11 further comprising testing for residual transmissible spongiform encephalopathy agent in contact with the object or material potentially contaminated with transmissible spongiform encephalopathy agent after contact with subtilisin protease.
